(19) United States
(12) Patent Application Publication
(10) Pub. No.: US 2026/0106002 A1
(43) Pub. Date: Apr. 16, 2026

(54) AUTOMATED COHORT IDENTIFICATION AND ASSEMBLY

(71) Applicant: Helix, Inc., San Mateo, CA (US)

(72) Inventors: Jui-Yi Hsieh, San Mateo, CA (US); Magnus Isaksson, Sunnyvale, CA (US)

(21) Appl. No.: 18/989,407

(22) Filed: Dec. 20, 2024

Related U.S. Application Data
(60) Provisional application No. 63/707,676, filed on Oct. 15, 2024.

(51) Int. Cl. G16H 10/60; G06F 16/334; G06F 16/35; G06F 16/901

(52) U.S. Cl. CPC G16H 10/60; G06F 16/3344; G06F 16/35; G06F 16/9024

(57) ABSTRACT

Systems and methods are provided for selecting cohorts of patients for study. One embodiment is a system that receives a request from a client for assembling a cohort of patients, and deploys a Large Language Model (LLM) that classifies the request into a target medical concept, consults a graph data structure that includes an entry for the target medical concept, and identifies additional medical concepts within a threshold distance of the target medical concept within the graph data structure. The system combines these, translates them into selection criteria for Electronic Health Record (EHR) data from a population, and adds patients from the population that meet the selection criteria into the cohort. The controller is further able to retrieve EHR data for each patient in the cohort, and to transmit the EHR data for the cohort to the client for review.

### FIELD

The disclosure relates to the field of health care, and in particular to the identification of patients having shared phenotypes for genetic research.

### BACKGROUND

Researchers desire tools for rapidly identifying and studying populations of patients who have similar conditions. However, researchers are required to maintain advanced and comprehensive knowledge of medical vocabulary codes in order to identify patients having the same condition. Existing medical vocabularies do not consistently report similar conditions using the same name or code, and also are not consistently used in the exact same manner by all health care providers. Hence, a single condition or set of related conditions may span tens of vocabulary codes, and the precise boundaries of the condition (e.g., using medical codes) may be unclear. This means that a direct code-based search is likely to fail to identify all of the patients that it may be desirable to study. It is also more difficult to prepare.

Patients and health care providers therefore continue to seek out new, robust solutions that are data-driven and consistent in identifying groups of related patients for treatment or study.

### SUMMARY

Embodiments described herein manage the selection of cohorts of patients for study, by reference to a graph data structure that indicates the relatedness of different medical concepts to each other. Specifically, the system utilizes a Large Language Model (LLM) which translates natural language provided by a user into medical concepts, and then refers to the graph data structure to expand search parameters for patients in a controlled manner. The combination of an LLM with a graph structure database in this manner helps to ensure that searches are both broad enough to include a wide swath of related patients, and precise enough to ensure that included patients exhibit similar phenotypes.

One embodiment is a system for selecting cohorts of patients for study. The system includes an interface configured to receive a request from a client for assembling a cohort of patients, and a controller able to deploy a Large Language Model (LLM) that classifies the request into a target medical concept, consults a graph data structure that includes an entry for the target medical concept, and identifies additional medical concepts within a threshold distance of the target medical concept within the graph data structure. The controller is further able to combine the target medical concept and the additional medical concepts into a combined set of medical concepts, to translate the combined set of medical concepts into selection criteria for Electronic Health Record (EHR) data from a population, and to add patients from the population that meet the selection criteria into the cohort. The controller is further able to retrieve EHR data for each patient in the cohort, and to transmit the EHR data for the cohort to the client for review.

A further embodiment is a method that includes receiving a request from a client for assembling a cohort of patients, classifying the request into a target medical concept via a Large Language Model (LLM), consulting a graph data structure that includes an entry for the target medical concept, and identifying additional medical concepts within a threshold distance of the target medical concept within the graph data structure. The method further includes combining the target medical concept and the additional medical concepts into a combined set of medical concepts, translating the combined set of medical concepts into selection criteria for Electronic Health Record (EHR) data from a population, adding patients from the population that meet the selection criteria into the cohort, retrieving EHR data for each patient in the cohort, and transmitting the EHR data for the cohort to the client for review.

A further embodiment is a non-transitory computer-readable medium storing instructions for performing a method. The method includes receiving a request from a client for assembling a cohort of patients, classifying the request into a target medical concept via a Large Language Model (LLM), consulting a graph data structure that includes an entry for the target medical concept, and identifying additional medical concepts within a threshold distance of the target medical concept within the graph data structure. The method further includes combining the target medical concept and the additional medical concepts into a combined set of medical concepts, translating the combined set of medical concepts into selection criteria for Electronic Health Record (EHR) data from a population, adding patients from the population that meet the selection criteria into the cohort, retrieving EHR data for each patient in the cohort, and transmitting the EHR data for the cohort to the client for review.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture in an illustrative embodiment.

FIG. 2 is a block diagram illustrating a genomics architecture in an illustrative embodiment.

FIG. 3 is a flowchart depicting a method for automatically generating and applying selection criteria to assemble a cohort of patients having similar medical conditions.

FIG. 4 is a flowchart depicting a method for processing natural language input in an illustrative embodiment.

FIG. 5 is a block diagram depicting a request transmitted to a genomics server for generating a cohort in an illustrative embodiment.

FIG. 6 depicts a graph data structure in an illustrative embodiment.

FIG. 7 depicts a selection of nodes within a graph data structure in an illustrative embodiment.

FIG. 8 depicts processing of natural language content from a request in an illustrative embodiment.

FIG. 9 is a block diagram depicting selection criteria for a cohort in an illustrative embodiment.

FIG. 10 is a block diagram that depicts summary statistics for a cohort in an illustrative embodiment.

FIG. 11 is a table that summarizes sequencing data for patients and is maintained at a genomics server in an illustrative embodiment.

FIG. 12 is a table that summarizes variant data for patients and is maintained at a genomics server in an illustrative embodiment.

FIG. 13 is a table that summarizes biomarker test data for patients and is maintained at a genomics server in an illustrative embodiment.

FIGS. 14-15 depict Graphical User Interfaces (GUIs) that facilitate the communication of information related to variant classifications in illustrative embodiments.

FIG. 16 depicts an illustrative computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

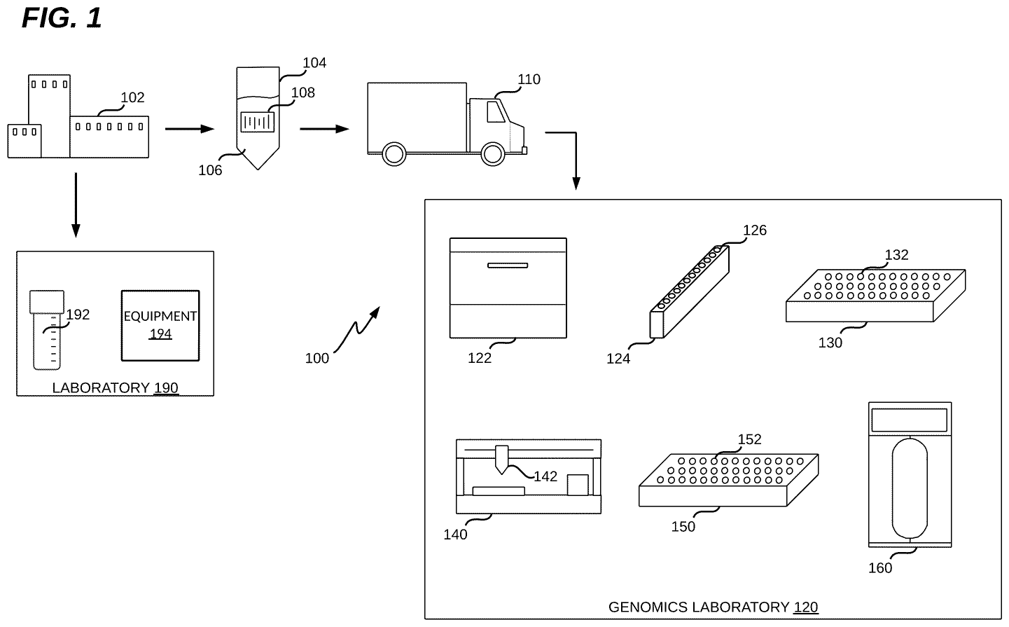

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Ribonucleic Acid (RNA) or Deoxyribonucleic Acid (DNA)) found within thousands or tens of thousands of samples 106 daily, via multiple healthcare provider networks 102.

Healthcare provider networks 102 may comprise hospitals, clinics, practitioner offices, laboratories, surgical centers, etc. that engage in or facilitate the practice of medicine. In one embodiment, healthcare provider networks 102 each comprise groups of hospitals that treat millions of patients. As a part of the practice of medicine, healthcare provider networks 102 acquire samples 106 for sequencing. For example, a healthcare provider network 102 may acquire samples 106 as part of a population screening program, as part of medical treatment, etc. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider networks 102 may also acquire samples 192 for conventional blood testing (described below). These samples 192 may be provided to laboratory 190 for analysis via equipment 194 (e.g., a chemically treated test strip, biochemical assay, etc.), or may be analyzed by patients via at-home testing methods. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although certain short-read technologies herein are discussed as utilizing hybridization capture techniques, amplicon-based techniques may be used alternatively or to supplement those techniques. Long-read techniques may also or alternatively be utilized.

### Accessioning

Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider network 102, etc. A CSI 108 may be reported via a barcode label, Quick Response (QR) code label, Radio Frequency Identifier (RFID) chip, or any suitable visual, transmission-generating, or other physical component affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as blood, saliva, mucus, etc. In some embodiments each of the samples 106 fills between two and five milliliters of volume within its corresponding sample container 104.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature, and duration of heating, may be chosen such that pathogenic material within the samples 106 is rendered harmless, or such that cellular lysis occurs. For example, heating may occur at a temperature of between forty and eighty (e.g., fifty) degrees Celsius (C), for a period of time between fifteen and two hundred (e.g., thirty) minutes. In some embodiments, including embodiments wherein the samples 106 are primarily the contents of a blood draw, the heating step may be foregone.

In this embodiment, upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine the CSI 108 for the sample 106, and may compare the CSI 108 to a CSI 108 listed on documentation provided in the external container. If there is a discrepancy between the CSI 108 on the sample container 104 and a CSI 108 listed in the documentation, the sample 106 may be flagged as having an error condition. Similarly, if the CSI 108 on the sample container 104 is damaged (e.g., abraded, heat-damaged, or water-damaged) and has become unreadable, the sample 106 may be flagged as having an error condition.

A technician or automated system may further inspect the contents of the sample container 104, via visual or other methods. If the sample 106 does not include expected constituent component (or is otherwise non-compliant) then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a genomics server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually distinct mark that enables rapid confirmation by a technician that the sample 106 has been integrated into the laboratory environment. The visually distinct mark may comprise a colored ring (e.g., around an entire perimeter of the sample container), a logo, a physical feature, a stamp, etc.

### Sample Plating

With the samples 106 having been successfully integrated into the environment of the genomics laboratory 120 environment, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System.” LH 140 may comprise, for example, a Hamilton Microlab Star Liquid Handling System.

In this embodiment, the LH 140 proceeds to transfer a portion of each sample 106 at a rack 124 to a well 132 within the sample microplate 130 that is not shared with other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a genomics server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

In embodiments wherein the genomics laboratory 120 performs both short-read and long-read sequencing workflows, the sample plating techniques discussed above may be performed separately, asynchronously, and/or in parallel for short-read technologies (e.g., via an Illumina sequencing platform such as a NovaSeq X) and for long-read technologies (e.g., via a PacBio sequencing patform such as a Revio). Samples 106 received at the genomics laboratory 120 may include sufficient genetic material to support multiple sequencing processes (e.g., both short-read and long-read sequencing processes).

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards. Specifically, storage enables re-sampling to occur in the event that there is a desire to re-sequence a sample 106.

### Extraction

Sample microplates 130 are transferred to a portion of the genomics laboratory 120 dedicated to extraction of the genetic material. The segment of the laboratory 120 that performs extraction and other pre-amplification operations may be sealed from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed wherein remaining fluid in each well is washed away. This ensures that potential impurities are removed from the well. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

In embodiments where the genomics laboratory 120 performs both short-read and long-read sequencing workflows, the extraction techniques discussed above may be performed separately, asynchronously, and/or in parallel for short-read technologies (e.g., via an Illumina sequencing platform such as a NovaSeq X) and for long-read technologies (e.g., via a PacBio sequencing patform such as a Revio).

### Library Preparation

After extraction is completed, library preparation may be performed for the contents of the genome stock microplate 150. The bead for each well, including ionically bonded genetic material, is transferred to a distinct well of a library preparation microplate (not shown). The library preparation microplate includes an identifier that uniquely distinguishes it from other library preparation microplates, and the LSI associated with each well on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, sealing the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment where short-read sequencing is performed, the pieces vary between three hundred and four hundred base pairs in length. These pieces are known as nucleic acid fragments. In a further embodiment where long-read sequencing is performed, the pieces may vary between five hundred and fifty thousand or more base pairs in length.

In one embodiment utilizing short-read sequencing, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library.”

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

In one embodiment utilizing long-read sequencing, library preparation may be performed via physical shearing of DNA to achieve a target size distribution mode between ten and twenty-five kilobases (kb), such as between fifteen and eighteen kb. The resulting nucleic acid fragments may be coupled to adapters to prepare them for sequencing via Single-Molecule Sequencing in Real Time (SMRT) or other long-read technologies.

The library preparation processes discussed herein may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the ease of sequencing desired portions of the genome. In some embodiments, enrichment is foregone for long-read sequencing processes.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing sequencing of all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques, via sequencing equipment 160 (e.g., an Illumina NovaSeq X sequencing machine, a PacBio Revio sequencing machine, etc.). As used herein, short-read sequencing refers to sequencing technologies that generate reads of less than five hundred base pairs in length. Short-read sequencing may be used as the basis for “synthetic long read” technologies that stitch individual short reads together, but as used herein, short-read sequencing does not refer to the creation or use of synthetic long reads.

In one embodiment, short-read sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

In a further embodiment, long-read sequencing (e.g., sequencing of nucleic acid fragments larger than one kilobase) is performed in a Single-Molecule Sequencing in Real Time (SMRT) process, wherein nucleic acid fragments are circularized and bound to a DNA polymerase enzyme. The bound pair enter a sequencing chamber, and the DNA polymerase adds complementary bases to the DNA strand that are fluorescently labeled to result in different colors for different bases.

As labelled bases are added by the polymerase, the color of the base is recorded, and then the fluorescent label is removed. The next base for the circularized nucleic acid fragment is then added and recorded, iteratively, until the circularized nucleic acid fragment has been sequenced a desired number of times.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

### Bioinformatics

Sequencing data may be stored in any suitable format. In one embodiment, raw sequencing data generated during short-read sequencing is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. In one embodiment, long-read sequencing data is output from the corresponding sequencing machine as one or more BAM files, obviating the need for long-read sequence data undergoing the conversion processes discussed above.

The aligned sequence data may then be called, resulting in a Variant Call Format (VCF) file reporting called variants at each location of the genome that was sequenced, together with secondary metrics such as quality indicator metrics. As used herein, a variant comprises a unique combination of genetic information, in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Genomics Architecture

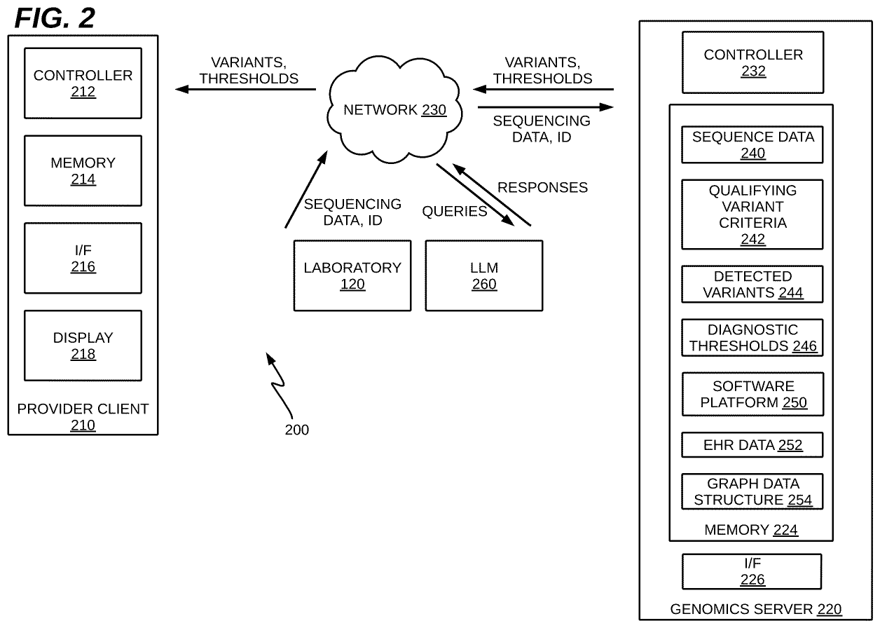

FIG. 2 is a block diagram illustrating a genomics architecture 200 in an illustrative embodiment. Genomics architecture 200 comprises any combination of systems and devices operable to review, process, and/or control access to sequencing data, including sequencing data received from genomics laboratory 120. In this embodiment, genomics architecture 200 comprises a genomics server 220 which receives sequencing data and identifiers (e.g., CSIs 108, LSIs, etc.) from genomics laboratory 120, via network 230. The sequencing data received and processed by the genomics server 220 may be supplied for multiple different types of sequencing operations, including short-read and long-read sequencing operations.

Genomics server 220 receives the sequencing data via interface (I/F) 226, such as an Ethernet interface, wireless interface compliant with Institute of Electrical and Electronics Engineers (IEEE) 802.11 standards, or other physical interface capable of transmitting and receiving digital data. The sequencing data 240 is stored in memory 224 for the population of patients (e.g., millions of patients) that have been sequenced by laboratory 120, and may be maintained in any suitable format. Examples of such formats include CRAM, VCF, BAM, and others. Memory 224 may store, for example, sequence data 240 describing multiple patients, and this sequence data 240 may be maintained in a de-identified format to facilitate the advancement of research. Memory 224 may be implemented via a cloud storage service, or may comprise a storage medium such as a hard disk or flash memory device.

Memory 224 may additionally store qualifying variant criteria 242, detected variants 244, and diagnostic thresholds 246 for diagnosis and/or treatment of specific diseases. In one embodiment, the portion of memory 224 storing these components is distinct from the portion of memory 224 storing sequence data 240.

Memory 224 further stores software platform 250 for directing interactions between users and an LLM 260. In one embodiment, the code for software platform 250 is maintained as code in javascript, Hypertext Markup Language (HTML) five, or other browser-friendly formats.

In some embodiments, the LLM 260 is integrated into software platform 250. Additionally, in some embodiments, software platform calls upon one or more LLMs 260 hosted by third parties available via network 230 (i.e., as depicted in FIG. 2). In particular, software platform 250 facilitates user interactions with LLM 260, further facilitates operations of the LLM 260 in accessing, analyzing, and/or building responses to queries, by reference to graph data structure 254.

Graph data structure 254 comprises a graph comprising nodes and edges/relationships, rather than in a relational database comprising structured tables with rows and columns. Graph data structure 254 includes nodes for multiple medical concepts, and aggregates content from one or more medical vocabularies (e.g., International Classification of Diseases (ICD), Current Procedural Terminology (CPT), Observational Medical Outcomes Partnership (OMOP) Common Data Model (CDM) vocabularies, and/or others), to facilitate rapid identification of related concepts. In one embodiment, graph data structure 254 has been refined to add edges/relationships representing inter-vocabulary relationships, such as those between diagnosing a medical concept (e.g., diabetes) in an ICD vocabulary, and treating for the medical concept in a CPT vocabulary.

In a further embodiment, memory 224 additionally stores Electronic Health Record (EHR) data 252 for one or more patients. The EHR data 252 may comprise records that have been rendered into a uniform format, such as an OMOP format, and may comprise health records for each patient that sequencing data has been stored for.

Controller 232 manages the operations of genomics server 220, and may for example analyze sequence data 240 to determine alignments to a reference genome, identify detected variants 244, control access and authentication related to sequence data 240, communicate with one or more provider clients 210, and/or perform additional operations. Controller 232 may be implemented, for example, as custom circuitry, as a hardware processor executing programmed instructions, as a combination of shared hardware processing resources implementing a compute service, or some combination thereof.

Genomics architecture 200 further comprises provider client 210, which is configured to permit users to interact with LLM 260 in order to generate cohorts. In some embodiments, provider client 210 is further configured to facilitate genomics-related activities, and receive information regarding detected variants 244 and/or diagnostic thresholds 246.

In the embodiment depicted in FIG. 2, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a projector, screen, etc. for presenting information to a user of provider client 210.

After sequencing data for a patient has been acquired, it is maintained at genomics server 220 in order to facilitate future studies associating relationships between genetic variants and phenotypes. This means that genomics server 220 has readily available access to clinic-genomic data sets that may be highly desirable for studies of cohorts of patients.

### Automated Cohort Selection

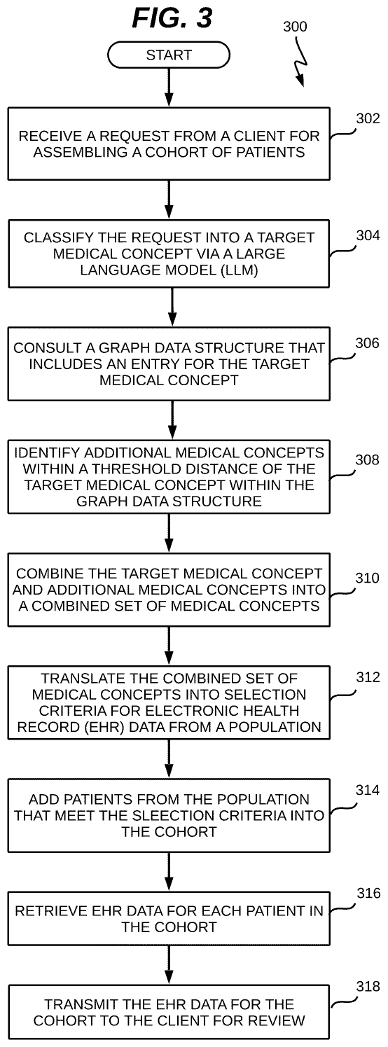

FIG. 3 is a flowchart depicting a method for automatically generating and applying selection criteria to assemble a cohort of patients having similar medical conditions. The steps of the flow charts described herein are not all inclusive and may include other steps not shown, and the steps may be performed in an alternative order. For example, method 300 may be performed serially or in parallel for each of multiple users accessing genomics server 220, which may host sequence data 240 and EHR data 252 for hundreds of thousands or millions of patients.

Assume, for this embodiment, that a user of a client, such as provider client 210, has logged into software platform 250 and has been authenticated. This may be performed for example, by provider client 210 provisioning a password and login to genomics server 220, exchanging an encrypted key with genomics server 220, etc. The user proceeds to interact with software platform 250 via the provider client 210, writing a query to indicate the nature of the desired cohort that the user would like to receive data for. However, the query is likely to include informal, nonspecific language, such as “blindness” or “anxiety”.

Step 302 comprises interface (I/F) 226 receiving a request from the client for assembling a cohort of patients. In many embodiments, the request includes a natural language description of the nature of the cohort. For example, the request may use plain language, such as that used by a layperson, to indicate desired conditions, and without resorting to references to specific medical codes, measurements, or other EHR-specific data points. The request may be decrypted by controller 232 upon receipt.

Step 304 comprises controller 232 utilizing software platform 250 to classify the request into at least one target medical concept, via LLM 260. In one embodiment, controller 232 directs LLM 260 to review natural language within the request in order to identify keywords associated with a list of medical concepts stored in memory. Based on the contents of the natural language, the LLM 260 finds one more target medical concepts. As used herein, a “medical concept” comprises a diagnosis, medical procedure, measurement (e.g., lab, vital, etc.), medication use, and/or exposure to a medical device. Hence, medical concepts comprise diseases, phenotypes, treatments, and/or conditions relating to medical care and treatment, such as those described in standard ontology systems. Examples of phenotypes for medical concepts may include “type II diabetes,” “obese,” or “skin cancer.” After the target medical concepts from the request have been identified, it remains desirable to operate LLM 260 to determine what pieces of content from EHR data 252 for patients could be used to identify patients having the target medical concepts. To achieve this goal, processing continues to steps 306-310, which may be repeated for each identified target medical concept.

Step 306 comprises the controller 232 operating the LLM 260 to consult a graph data structure 254 that includes an entry for the target medical concept. In one embodiment, this comprises the LLM 260 comparing the target medical concepts to the contents of nodes within the graph data structure 254. In the event of a high-confidence match, such as a match between the medical concept and the contents of a node (e.g., at a confidence determined by the LLM of “high” or greater than ninety percent), the LLM 260 selects the node for inclusion in selection criteria.

In one embodiment, the controller 232 performs a direct similarity comparison between the target medical concept and nodes in the graph data structure, and returns (or selects) a top N number of nodes (e.g., one node, three nodes, or ten nodes, etc.) corresponding with the target medical concept. This may be performed by calculating either the cosine distance or the Euclidean distance between a vectorized target medical concept and each of the nodes. The LLM 260 facilitates comprehension of the request, by vectorizing natural language within the request as part of processing.

In a further embodiment, the LLM 260 is granted the freedom to decide what to do. This may be performed as an alternative or a supplement to the direct similarity comparisons discussed above. For example, the LLM 260 may be prompted to use its own approach and may take the same approach as a first approach, or may decide to match not only the nodes but also a path (i.e., a pattern of connections between nodes, such as from drug_name-treats to condition_name). In this approach, the LLM 260 returns not only similar nodes to the target medical concept but also nodes related to those similar nodes. In a further example, the LLM 260 is given access to a schema of the graph data structure, and is allowed to freely and autonomously decide what nodes and relationships to use/retrieve.

Step 308 comprises the controller 232 operating the LLM 260 to identify additional medical concepts within a threshold distance of the target medical concept within the graph data structure 254. That is, for each node in the graph data structure 254 that was selected in step 306, the controller 232 identifies additional nodes that are within a threshold number of “steps” (e.g., two steps, three steps, etc.) of the selected nodes within the graph data structure 254. The additional nodes are selected, and medical concepts recited in the additional nodes are chosen as additional medical concepts.

Step 310 comprises controller 232 combining the target medical concept and the additional medical concepts into a combined set of medical concepts. This may comprise controller 232 operating the LLM 260 to recite each of the target medical concepts and the additional medical concepts as part of a uniform list, or may comprise controller 232 utilizing code for software platform 250 to perform this function.

Step 312 comprises controller 232 translating the combined set of medical concepts into selection criteria for EHR data 252 for a population. This may comprise identifying medical vocabulary codes, measurements, lab results, or conditions identified within each selected node of the graph data structure 254, and adding those items to search criteria for EHR data 252.

In one embodiment, each node of graph data structure 254 for a concept further recites a medical vocabulary code, measurement, lab result, or condition which is reportable in an EHR. In embodiments where one or more nodes of graph data structure define EHR-reportable content, controller 232 may identify this content as applicable to the cohort. Controller 232 proceeds to compile these items together (e.g., using logical OR or AND statements, as indicated by the natural language) to prepare the search criteria. In a further embodiment, nodes of the graph data structure for target medical concepts are neighbored by additional nodes, such as nodes reporting phecodes (e.g., combinations of phenotypes), custom codesets across one or more medical vocabularies, publication data, internal research data, patient nodes, and/or more. Thus, in some embodiments graph data structure 254 is more than a union of medical-concept listings.

After step 312 has been completed, in one embodiment controller 232 transmits a message to provider client 210 indicating the selection criteria, after which the provider client 210 may update a screen or GUI to present the selection criteria to the user. The user may then accept the selection criteria, provide a natural language update for processing by LLM 260 to revise the selection criteria, or provide a new request to prompt the controller 232 to restart the cohort selection process of method 300. In a further embodiment, the GUI permits the user to confirm and reject individual selection criterion. Those selection criteria which are rejected are then removed by controller 232 from the selection criteria before proceeding to step 314.

Step 314 comprises controller 232 adding patients from the population that meet the selection criteria into the cohort. In one embodiment, step 314 comprises controller 232 reviewing the EHR data 252 for patients in the population (e.g., via database operations) to detect the presence of those specific items. In the event that desired items are included, and undesirable items are not included, in the EHR data 252 for a patient, the patient is included in the cohort. When a patient is selected, controller 232 may update a list of unique patient identifiers for the cohort to include that patient.

Step 316 comprises controller 232 retrieving EHR data 252 for each patient in the cohort, and step 318 comprises I/F 226 transmitting the EHR data 252 for the cohort to the provider client 210 for review. In many embodiments, controller 232 provides EHR data 252 in the form of summary statistics, or in an otherwise de-identified format. In further embodiments, controller 232 provides EHR data 252 within a limited range of time (e.g., within ten years before and/or after) a patient met the selection criteria created by the LLM 260, or a limited range of types of information (e.g., solely information relating to cardiac health, etc.). The user, upon receiving the EHR data 252 for the cohort, may then load the EHR data 252 into provider client 210 for further study.

In a further embodiment, controller 232 additionally operates LLM 260 to translate the combined set of medical concepts into additional selection criteria for sequence data 240 for the population. That is, the selection criteria may be specific to genetic conditions that are not reported (or widely reported) within the EHR data 252 for the population.

Controller 232 may translate the combined set of medical concepts into the additional selection criteria by reference to graph data structure 254, in embodiments where graph data structure 254 includes medical concepts related to the interpretation or analysis of sequence data 240 (e.g., variant calls such as those from VCF files, variant classifications under American College of Medical Genetics (ACMG) guidelines, etc.). Alternatively, medical concepts related to the interpretation or analysis of sequence data 240 may be stored in a separate graph data structure maintained within memory 224, that is specifically targeted to medical concepts in genomics.

Controller 232 may format the additional selection criteria into a similar format as the selection criteria for EHR data 252, or may alternatively utilize a unique format. For example, controller 232 may apply genetic criteria by reference to default/minimum values for Phred Quality scores (e.g., a Phred Quality score of 20), to reference specific classifications of variants under ACMG guidelines (e.g., benign, likely benign, Variant of Unknown Significance (VUS), likely pathogenic, pathogenic, etc.), by reference to specific genes associated with the combined medical concepts, or via other standardization and classification techniques upon plain language references to medical concepts that are related to genetic conditions.

Controller 232 further reviews the sequence data 240 to identify patients meeting the additional selection criteria, and adds patients from the population that meet the additional selection criteria into the cohort. In one embodiment, controller 232 performs these operations by reference to compiled variant classification data for the patients, variant calls for the patients, or other genetic information stored in genomics server 220.

Method 300 provides a notable technical benefit via the combination of a specifically populated graph data structure for medical concepts into the operation of an LLM, in order to rapidly, accurately and expansively identify selection criteria for cohorts of patients. This effectively expands search criteria for a cohort in a controlled manner, without requiring substantial investments in time and labor by a user. Specifically, because medical concepts are maintained in a novel graph data structure wherein each node ties a medical concept to a specific piece of information that could be identified in an EHR (and/or in sequence data), an LLM is able to rapidly acquire selection criteria for a concept. Additionally, because the graph data structure connects nodes by relationship, the identification of medical concepts that are related is highly processing-efficient.

Put together, this unique arrangement of architectural components (i.e., LLM technology and a graph data structure maintaining specific pieces of information organized in a specific manner) enables highly effective searches, without requiring users to carefully consult lengthy vocabulary code sets or specific medical definitions. By using an LLM as an intermediary to interpret these requests, users may now spend more time reviewing and revising cohort selection criteria than attempting to write those selection criteria from scratch.

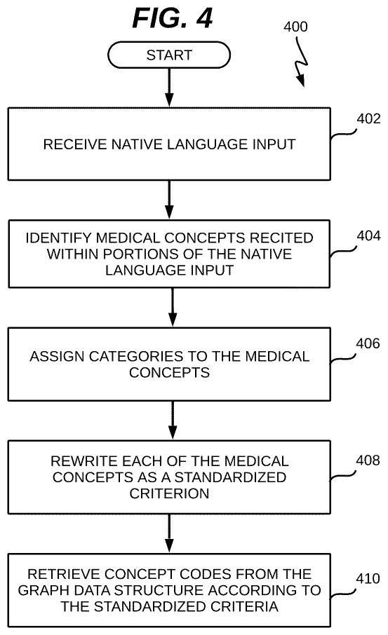

FIG. 4 is a flowchart depicting a method 400 for processing natural language input in an illustrative embodiment. Method 400 may be performed, for example, during steps 304-306 of method 300.

Step 402 comprises I/F 226 receiving natural language input as a part of a query from the user. For example, the natural language input may comprise plain text or rich text, stored within a text field of the request. The natural language input includes text referring to one or more medical concepts, but need not refer to specific medical vocabulary codes, measurements, laboratory results, diseases or conditions associated with those medical concepts.

Step 404 comprises controller 232 operating LLM 260 to identify medical concepts recited within portions of the native language input. This may comprise, for example, controller 232 instructing LLM 260 to identify medical concepts that have a high confidence relationship to a specific entry or node within the graph data structure 254, as the graph data structure 254 stores medical concepts. In one embodiment, this comprises LLM 260 comparing words and phrases within the natural language input to words and phrases recited in medical concepts. In the event that the comparison results in more than a threshold level of confidence (e.g., self-reporting by the LLM 260 of a “high” level of confidence that a phrase is the same as a medical concept reported in a node), the medical concept is identified. In a further embodiment, the LLM 260 vectorizes the natural language input and/or phrase being considered, and controller 232 compares this vectorized content to vectorized versions of medical concepts maintained in memory 224.

Controller 232 may operate LLM 260 to identify multiple medical concepts within the same natural language input. In a further embodiment, controller 232 instructs LLM 260 to identify not just medical concepts, but also values associated with those concepts. For example, a medical concept for blood pressure may be assigned a desired range of values of greater than 120 mmHg within the natural language input. The LLM 260 therefore operates to identify the corresponding value within the natural language input, and to associate it with the medical concept.

Step 406 comprises controller 232 operating the LLM 260 to assign categories to the medical concepts. In one embodiment, the categories include medical conditions (e.g., diseases, information related to organ function (such as bradycardia), etc.), laboratory tests, medical procedures, demographics, and measurements. Assigning categories to medical concepts has notable value. For example, certain medical concepts, or entire categories of medical concepts such as certain laboratory tests, medical procedures, demographics, and/or measurements, may not be represented in graph data structure 254, will still be relevant for analysis of the EHR data 252. In one embodiment, controller 232 foregoes concept retrieval for such categories and/or specific medical concepts.

Categorization is also valuable because it aids controller 232 in operating the LLM 260. For example, measurements and laboratory tests may be most relevant for cohort selection when they are taken prior to formal diagnosis of a patient for a specific medical condition. For example a patient diagnosed with diabetes may be prescribed a medication to lower their blood sugar, meaning that their blood sugar levels after diagnosis may be similar to that of a typical member of the population. This would make blood sugar levels, post-diagnosis, a poor selection criteria for finding patients with diabetes.

Step 408 comprises controller 232 operating the LLM to rewrite each of the medical concepts as a standardized criterion. In one embodiment, a standardized criterion clearly recites the medical concept, followed by any values within the natural language input associated with the medical concept (e.g., based on sentence structure and/or units of measurement). For example, a standardized criterion may indicate “DIABETES: PRESCRIBED SGLT2 INHIBITOR, BLOOD SUGAR<100 mg/dL AFTER MEDICATION PRESCRIBED.” Separate criteria may then be delineated with a line break, field tag, or similar item.

Step 410 comprises controller 232 retrieving concept codes from the graph data structure 254 according to the standardized criteria defined in step 408. That is, nodes within the graph data structure 254 which are associated with a standardized criteria are selected, such as concept codes associated with diabetes in the example above. In some embodiments, controller 232 refrains from operating LLM 260 to review graph data structure 254 for certain categories of medical concepts. For example, in one embodiment LLM 260 restricts itself to only mapping medical conditions to nodes within graph data structure 254.

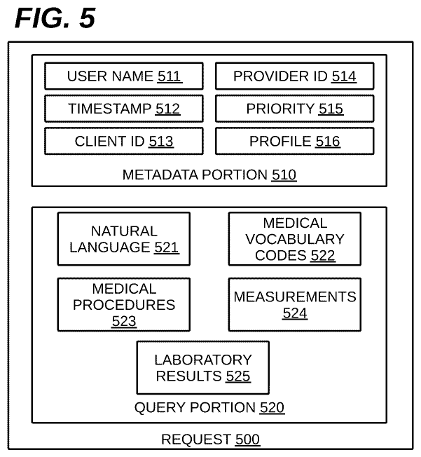

With various methods described above relating to LLM interactions with graph data structures in order to select patients for a cohort, the following FIGS. 5-13 provide context into the arrangement and format of various data structures in illustrative embodiments.

FIG. 5 is a block diagram depicting a request 500 transmitted to a genomics server for generating a cohort in an illustrative embodiment. Assume, for this embodiment, that request 500 has been generated by provider client 210 in response to user input, and has been transmitted to I/F 226 of genomics server 220. In this embodiment, request 500 comprises more than just a natural language query for cohort selection. Specifically, request 500 includes a metadata portion 510, which provides contextual information for the request 500. Examples of contextual information include a user name 511 for the user who is generating the request, a timestamp 512 at which the request was generated or transmitted, a client identifier (ID) 513 for the client that is generating the request, and a provider ID 514 for the healthcare provider or network associated with the user. The metadata portion 510 may further include a priority 515 of the request 500, indicating how the request should be queued. For example, priorities may be set to “high”, “medium”, or “low”, and queued at genomics server 220 such that high priority requests are processed more quickly than (e.g., with a lower permitted maximum wait time) or before medium priority requests, which are processed more quickly than or before low priority requests.

Metadata portion 510 may further include a profile 516 for the user, and this profile information may indicate preferences of the user. In one embodiment, the preferences include header or footer content to add before or after the natural language portion of the request, for handling by a Large Language Model (LLM). For example, header information for a profile may indicate that the user's focus is in the field of cardiology, that the user prefers a certain threshold number of steps (e.g., four steps) when traversing the graph data structure, or that the user wishes to create a cohort of patients solely from a specific health care network. This profile information helps to ensure that the LLM 260 generates content that is suitable to the preferences of the user.

Request 500 further includes a query portion 520. The query portion 520 includes natural language 521 defining parameters for a requested cohort. The natural language 521 may be accompanied by medical vocabulary codes 522, medical procedures 523, measurements 524, and/or laboratory results 525.

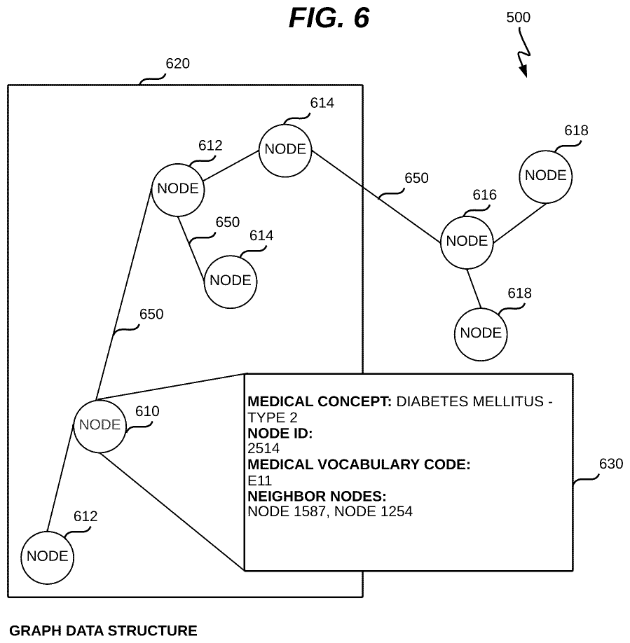

FIG. 6 depicts a graph data structure 600 in an illustrative embodiment. Graph data structure 600 is a simplified version of a graph data structure 254, provided to enhance conceptual understanding of nodes, the contents of nodes, and edges between nodes. In this embodiment, graph data structure 600 includes a node 610 which corresponds with a medical concept identified from within a query. A controller 232 selects all nodes within a threshold distance of two steps of the node 610. This means that nodes 612 that are one step from node 610, and nodes 614 that are two steps from node 610, are included within selection criteria 620. Node 616 and nodes 618 are not selected. Distances are determined by calculating the number of edges 650 between nodes. In this embodiment, each node includes its own content 630, which stores information identifying neighbor nodes, identifying the current node, and reciting a medical vocabulary code, medical concept, laboratory test, and/or measurement for the node.

Graph data structure 600 provides a unique architecture for storing medical concept data, by tying specific nodes and concepts to specific types of EHR-linked content. This enables a concept to be directly mapped to specific portions of EHR data, even portions of EHR data that are maintained within free-text fields. This facilitates the operation of an LLM to identify desired portions of content that correspond to specific medical concepts within EHR data 252.

In further embodiments, the building of cohorts by reference to a graph data structure may be validated with, or supplemented by, the process of vectorizing patient information, such as via the processes described in “Phe2vec: Automated disease phenotyping based on unsupervised embeddings from electronic health records,” De Freitas, Jessica K. et al., Patterns, Volume 2, Issue 9, 100337, herein incorporated by reference. Comparison of patients within a vectorized space may facilitate the identification of similar patients in a manner that helps to ensure the accuracy and breadth of the graph data structure techniques described herein.

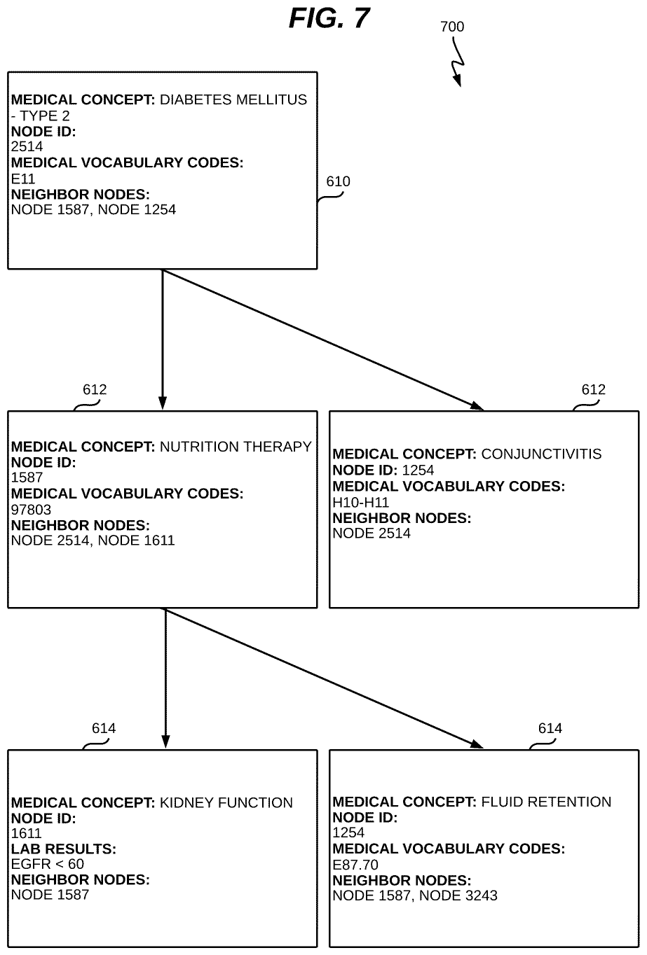

FIG. 7 depicts a selection 700 of nodes within a graph data structure in an illustrative embodiment. Specifically, FIG. 7 depicts the same selection of nodes shown in FIG. 6 for graph data structure 600. As shown in FIG. 7, nodes 612 are within one step of node 610, and nodes 614 are within two steps of node 610. Each node includes its own content reciting information relevant to a medical concept of interest.

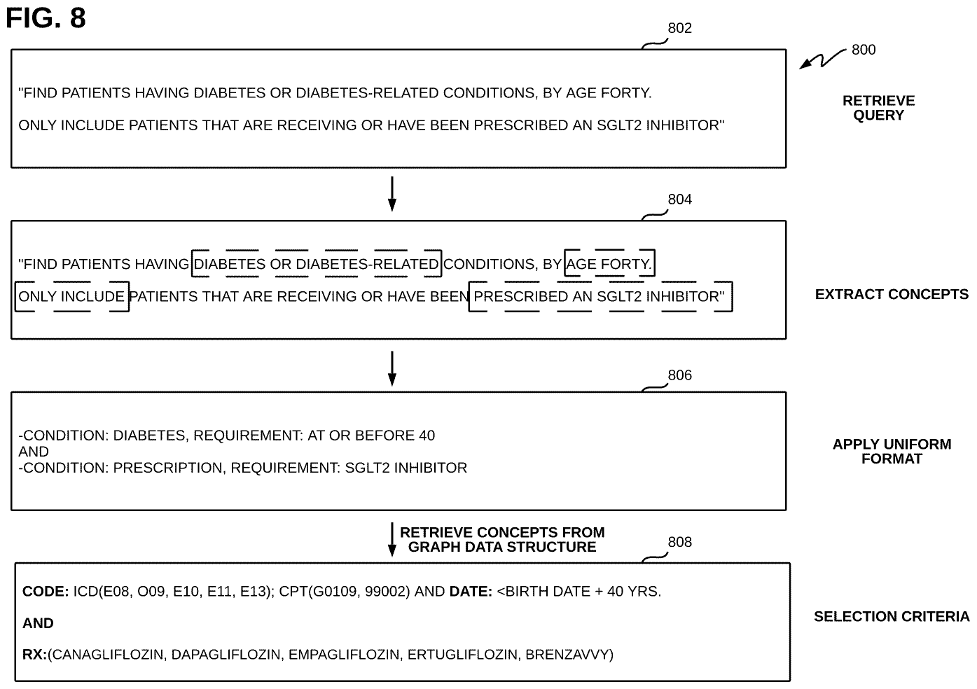

FIG. 8 is a diagram 800 that depicts processing of natural language content from a request in an illustrative embodiment. As shown in FIG. 8, natural language 802 is retrieved from a request by an LLM 260. The LLM extracts concepts from the natural language by phrases 804 that correspond with medical conditions, measurements, or laboratory procedures. The LLM then processes the extracted concepts into a uniform format 806. In this embodiment, the uniform format indicates the name of each concept, followed by values required for each concept. Next, the LLM 260 retrieves the concepts from the graph data structure to generate a set of selection criteria 808.

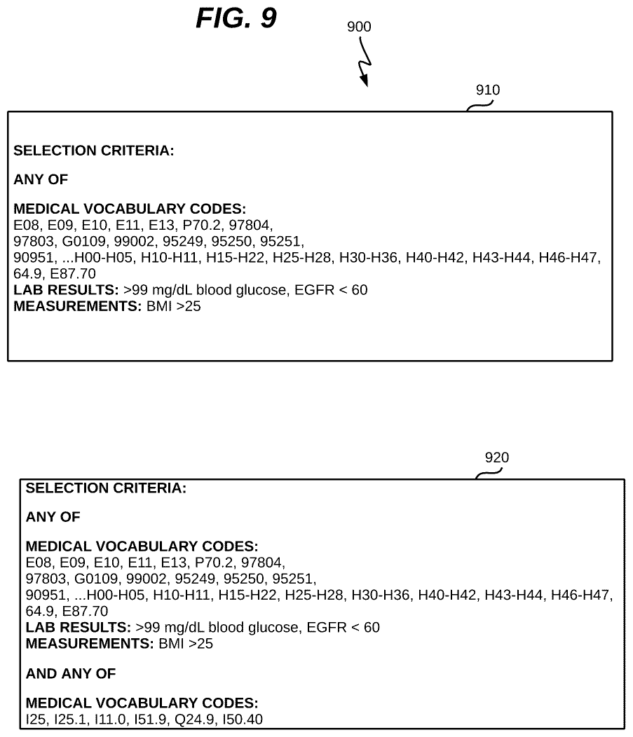

FIG. 9 is a block diagram 900 depicting selection criteria for a cohort in an illustrative embodiment. Specifically, FIG. 9 depicts selection criteria 910 as well as selection criteria 920. Selection criteria 910 indicates that any patient having a specific medical vocabulary code, laboratory result (e.g., prior to receiving medication related to the medical concept), or measurement (e.g., prior to receiving medication related to the medical concept) qualifies for the cohort.

Selection criteria 920 selects only patients that have both one of a wide range of medical codes, as well as one of a narrow range of medical codes. This facilitates precision cohort selection, and enables a user to consider patients having multiple different types of medical conditions.

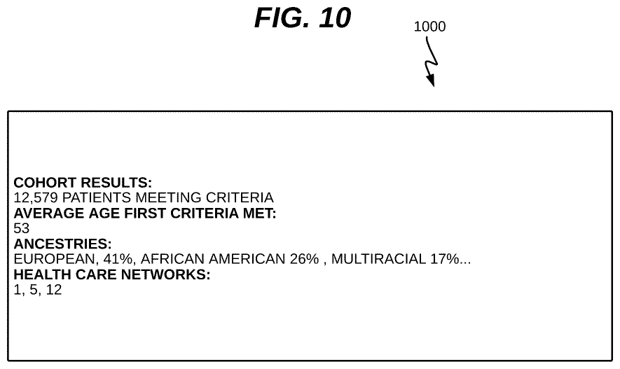

FIG. 10 is a block diagram 1000 that depicts a cohort summary in an illustrative embodiment. The cohort summary recites a number of patients in the cohort, an age at which patients in the cohort typically met the criteria for inclusion, demographic information for patients in the cohort, and a listing of health care networks that the patients were drawn from.

In further embodiments, medical concepts include genetic testing results, and graph data structure 254 includes medical concepts for genetic testing results, such as variant classifications, carrier status for pathogenic variants, and other reporting information, on a patient-by-patient basis for patients reported by the EHR data 252. Hence, in some embodiments graph data structure 254 exhibits an even more unique architecture, in the form of a combination of nodes that consider both EHR-linked medical concepts and genetic testing-linked medical concepts. This enables a user to build a cohort of patients not just by reference to EHR data 252, but also by reference to sequence data 240. In short, this arrangement provides a notable technical benefit by permitting selection of a cohort of patients based on clinicogenomic criteria.

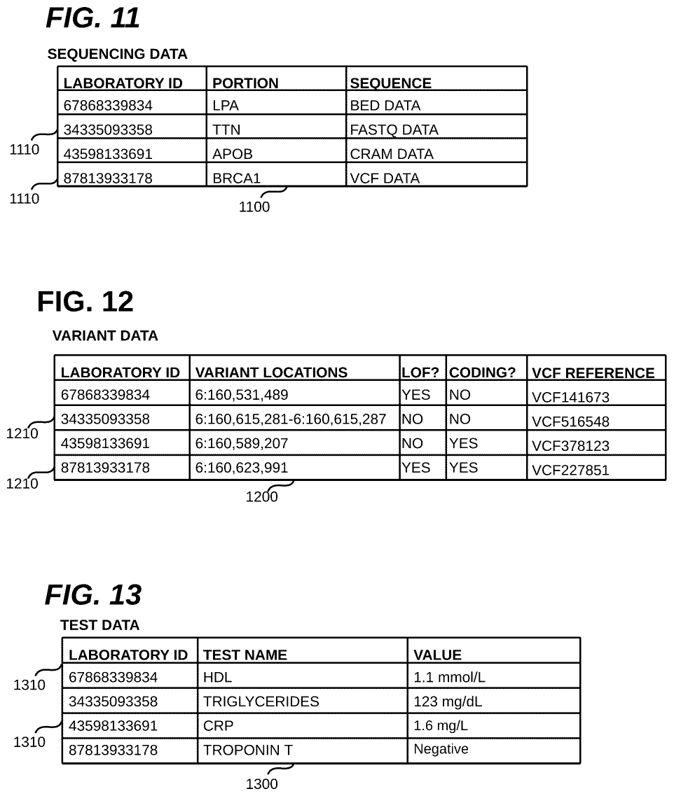

FIG. 11 is a table 1100 that summarizes sequencing data for one or more genes for individuals in an illustrative embodiment. For example, table 1100 may be one of many data structures stored in genomics server 220. In this embodiment, table 1100 includes an entry 1110 for each of multiple patients. Each entry 1110 includes a unique identifier (e.g., LSID) for the corresponding patient, as well as an indication of the gene that the sequence data relates to. The portion of the genome that has been sequenced may comprise whole genome data, whole exome data, array data, data for a specific gene or portion of a gene, etc. Table 1100 also indicates a format of the sequence data. Table 1100 may be generated based on, or with reference to, sequences that have been alignment-enhanced via the processes discussed above.

FIG. 12 is a table 1200 that summarizes variant data for individuals in an illustrative embodiment. In this embodiment, each entry 1210 in table 1200 reports a location (e.g., chromosomal coordinate) for each genetic variant, together with flags indicating whether the variant is a Loss of Function (LoF) variant or a coding variant. Table 1200 further includes a VCF reference, which refers to the location and/or identifier of a VCF file that indicates the presence of the variant. The VCF file may be generated using data from the alignment enhancement processes discussed above. For example, alignment-enhanced data in a BAM, SAM, or CRAM file may include data used to generate the VCF file. Table 1200 may be utilized by controller 232 of genomics server 220, in order to rapidly select and report diagnostic and treatment thresholds for a patient. Table 1200 may be generated based on, or with reference to, sequences that have been alignment-enhanced via the processes discussed above.

FIG. 13 is a table 1300 that summarizes biomarker test data for individuals in an illustrative embodiment. Specifically, table 1300 summarizes test data pertaining to predetermined diseases for each of multiple patients in an illustrative embodiment. Each entry 1310 in table 1300 indicates an anonymized laboratory ID for a patient, a corresponding test name, and a corresponding value. Table 1300 may be created, for example, based on EHR data retrieved for patients. Laboratory IDs may be associated with EHR identifiers at genomics server 220 or provider client 210, in order to enable access to both health data and genomics data for a patient. Table 1300 may be used to enhance or provide context for genetic insights determined based sequences that have been alignment-enhanced via the processes discussed above.

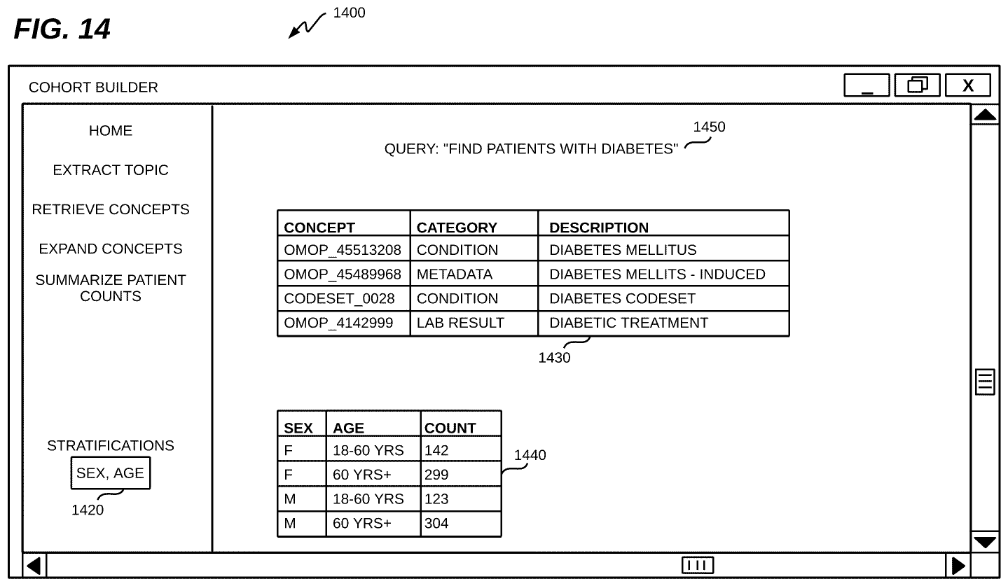

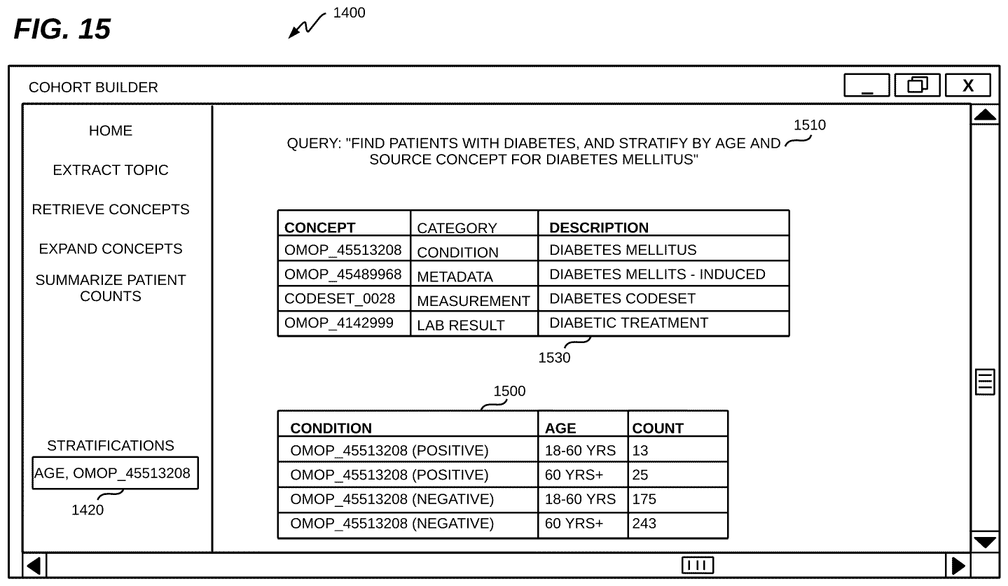

FIGS. 14-15 depict Graphical User Interfaces (GUIs) that facilitate the communication of information related to cohort building in illustrative embodiments. These GUIs may be presented, for example, via a browser window or other portion of a screen of one or more provider clients 210.

FIG. 14 depicts a GUI 1400 that reports a received natural language query 1450, together with selection criteria 1430 determined from the natural language query 1450. GUI 1400 further includes element 1440, which reports out stratified summary metrics 1440 for the cohort identified using the selection criteria 1430, and a selection key 1420 for stratification dimensions. In this embodiment, GUI 1400 which stratifies the summary statistics by age and sex.

FIG. 15 depicts the GUI 1400 of FIG. 14, with summary statistics stratified by age and a selected medical concept. In FIG. 15, the natural language query 1510 itself includes language requesting stratification of patients within the cohort. In this embodiment, controller 232 operates the LLM 260 to review and identify the stratifications, for presentation in GUI 1400. Thus, GUI 1400 includes new stratification summary statistics 1500, which may be based on the natural language query 1510 and/or manual use of the selection key 1420 by a user.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as genomics server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by an entity. Cloud computing relies on the sharing of resources, and generally includes on-demand self-service, broad network access, resource pooling, rapid elasticity, and measured service.

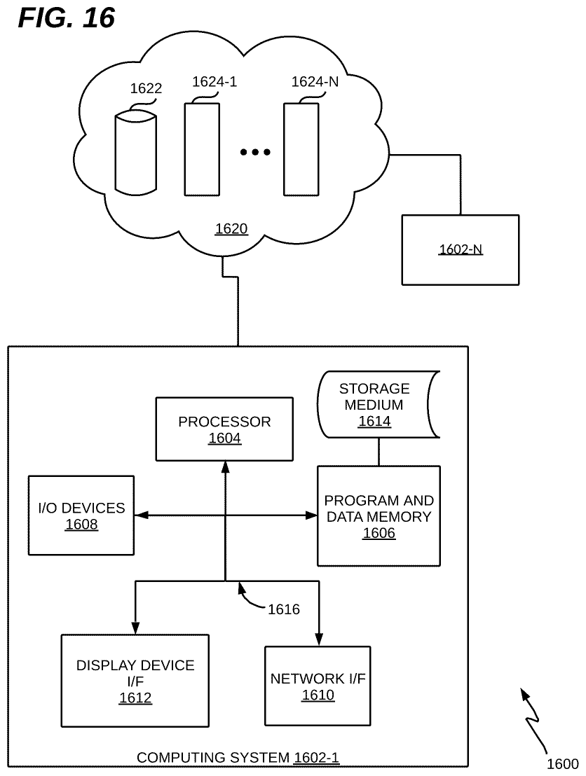

FIG. 16 depicts one illustrative cloud computing system 1600 operable to perform the above operations by executing programmed instructions tangibly embodied on one or more computer readable storage mediums. The cloud computing system 1600 generally includes the use of a network of remote servers hosted on the internet to store, manage, and process data, rather than a local server or a personal computer (e.g., in the computing systems 1602-1-1602-N). Cloud computing enables users to use infrastructure and applications via the internet, without installing and maintaining them on-premises. In this regard, the cloud computing network 1620 may include virtualized information technology (IT) infrastructure (e.g., servers 1624-1-1624-N, the data storage module 1622, operating system software, networking, and other infrastructure) that is abstracted so that the infrastructure can be pooled and/or divided irrespective of physical hardware boundaries. In some embodiments, the cloud computing network 1620 can provide users with services in the form of building blocks that can be used to create and deploy various types of applications in the cloud on a metered basis.

Various components of the cloud computing system 1600 may be operable to implement the above operations in their entirety or contribute to the operations in part. For example, a computing system 1602-1 may be used to perform analysis of gene sequencing data, and then store that analysis along with the gene sequencing data in a data storage module 1622 (e.g., a database) of a cloud computing network 1620. Various computer servers 1624-1-1624-N of the cloud computing network 1620 may be used to operate on the gene sequencing data and/or transfer the gene sequencing analysis and/or the gene sequencing data to another computing system 1602-N.

Some embodiments disclosed herein may utilize instructions (e.g., code/software) accessible via a computer-readable storage medium for use by various components in the cloud computing system 1600 to implement all or parts of the various operations disclosed hereinabove. Examples of such components include the computing systems 1602-1-1602-N.

Exemplary components of the computing systems 1602-1-1602-N may include at least one processor 1604, a computer readable storage medium 1614, program and data memory 1606, input/output (I/O) devices 1608, a display device interface 1612, and a network interface 1610. For the purposes of this description, the computer readable storage medium 1614 comprises any physical media that is capable of storing a program for use by the computing system 1602. For example, the computer-readable storage medium 1614 may be an electronic, magnetic, optical, electromagnetic, infrared, semiconductor device, or other non-transitory medium. Examples of the computer-readable storage medium 1614 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 1604 is coupled to the program and data memory 1606 through a system bus 1616. The program and data memory 1606 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 1608 (including but not limited to keyboards, displays, touchscreens, microphones, pointing devices, etc.) may be coupled either directly or through intervening I/O controllers. Network adapter interfaces 1610 may also be integrated with the system to enable the computing system 1602 to become coupled to other computing systems or storage devices through intervening private or public networks. The network adapter interfaces 1610 may be implemented as modems, cable modems, Small Computer System Interface (SCSI) devices, Fibre Channel devices, Ethernet cards, wireless adapters, etc. Display device interface 1612 may be integrated with the system to interface to one or more display devices, such as screens for presentation of data generated by the processor 1604.

What is claimed is:

1. A system for selecting cohorts of patients for study, the system comprising:
an interface configured to receive a request from a client for assembling a cohort of patients; and
a controller configured to deploy a Large Language Model (LLM) that classifies the request into a target medical concept, consults a graph data structure that includes an entry for the target medical concept, and identifies additional medical concepts within a threshold distance of the target medical concept within the graph data structure,
the controller being further configured to combine the target medical concept and the additional medical concepts into a combined set of medical concepts, to translate the combined set of medical concepts into selection criteria for Electronic Health Record (EHR) data from a population, and to add patients from the population that meet the selection criteria into the cohort, and
the controller being further configured to retrieve EHR data for each patient in the cohort, and to transmit the EHR data for the cohort to the client for review.

2. The system of claim 1 wherein:
the selection criteria require at least one item selected from the group consisting of: a medical vocabulary code; a lab result; and a measurement, and the selection criteria further require that the at least one item is associated with the combined set of medical concepts.

3. The system of claim 1 wherein:
nodes within the graph data structure represent medical concepts; and
the LLM consults the graph data structure by identifying additional nodes within a threshold number of edges of a node for the target medical concept within the graph data structure.

4. The system of claim 3 wherein:
each node within the graph data structure identifies a different medical concept; and
each node within the graph data structure is associated with one or more items selected from the group consisting of: a medical vocabulary code; a lab result; and a measurement.

5. The system of claim 1 wherein:
the request comprises a free text field; and
the LLM processes the free text field to select the target medical concept.

6. The system of claim 1 wherein:
the EHR data from the population comprises medical vocabulary codes, lab results, and measurements corresponding with the target medical concept and the additional medical concepts, on a patient-by-patient basis.

7. The system of claim 1 wherein:
the controller is further configured to translate the combined set of medical concepts into additional selection criteria for sequencing data for the population, to review the sequencing data to identify patients meeting the additional selection criteria, and to add patients from the population that meet the additional selection criteria into the cohort.

8. A method comprising:
receiving a request from a client for assembling a cohort of patients;
classifying the request into a target medical concept via a Large Language Model (LLM);
consulting a graph data structure that includes an entry for the target medical concept;
identifying additional medical concepts within a threshold distance of the target medical concept within the graph data structure;
combining the target medical concept and the additional medical concepts into a combined set of medical concepts;
translating the combined set of medical concepts into selection criteria for Electronic Health Record (EHR) data from a population;
adding patients from the population that meet the selection criteria into the cohort;
retrieving EHR data for each patient in the cohort; and
transmitting the EHR data for the cohort to the client for review.

9. The method of claim 8 wherein:
the selection criteria require at least one item selected from the group consisting of: a medical vocabulary code; a lab result; and a measurement, and the selection criteria further require that the at least one item is associated with the combined set of medical concepts.

10. The method of claim 8 wherein:
nodes within the graph data structure represent medical concepts; and
consulting the graph data structure comprises identifying additional nodes within a threshold number of edges of a node for the target medical concept within the graph data structure.

11. The method of claim 10 wherein:
each node within the graph data structure identifies a different medical concept; and
each node within the graph data structure is associated with one or more items selected from the group consisting of: a medical vocabulary code; a lab result; and a measurement.

12. The method of claim 8 wherein:
the request comprises a free text field; and
classifying the request comprises processing the free text field to select the target medical concept.

13. The method of claim 8 wherein:
the EHR data from the population comprises medical vocabulary codes, lab results, and measurements corresponding with the target medical concept and the additional medical concepts, on a patient-by-patient basis.

14. The method of claim 8 further comprising:
translating the combined set of medical concepts into additional selection criteria for sequencing data for the population;
reviewing the sequencing data to identify patients meeting the additional selection criteria; and
adding patients from the population that meet the additional selection criteria into the cohort.

15. A non-transitory computer readable medium embodying programmed instructions which, when executed by a processor, are operable for performing a method for enhancing sequencing data, the method comprising:
receiving a request from a client for assembling a cohort of patients;
classifying the request into a target medical concept via a Large Language Model (LLM);
consulting a graph data structure that includes an entry for the target medical concept;
identifying additional medical concepts within a threshold distance of the target medical concept within the graph data structure;
combining the target medical concept and the additional medical concepts into a combined set of medical concepts;
translating the combined set of medical concepts into selection criteria for Electronic Health Record (EHR) data from a population;
adding patients from the population that meet the selection criteria into the cohort;
retrieving EHR data for each patient in the cohort; and
transmitting the EHR data for the cohort to the client for review.

16. The medium of claim 15 wherein:
the selection criteria require at least one item selected from the group consisting of: a medical vocabulary code; a lab result; and a measurement, and the selection criteria further require that the at least one item is associated with the combined set of medical concepts.

17. The medium of claim 15 wherein:
nodes within the graph data structure represent medical concepts; and
consulting the graph data structure comprises identifying additional nodes within a threshold number of edges of a node for the target medical concept within the graph data structure.

18. The medium of claim 17 wherein:
each node within the graph data structure identifies a different medical concept; and
each node within the graph data structure is associated with one or more items selected from the group consisting of: a medical vocabulary code; a lab result; and a measurement.

19. The medium of claim 15 wherein:
the request comprises a free text field; and
classifying the request comprises processing the free text field to select the target medical concept.

20. The medium of claim 15 wherein:
the EHR data from the population comprises medical vocabulary codes, lab results, and measurements corresponding with the target medical concept and the additional medical concepts, on a patient-by-patient basis.
